(19) United States
(12) Patent Application Publication
(10) Pub. No.: US 2026/0207479 A1
(43) Pub. Date: Jul. 23, 2026

(54) SINGLE VIAL VALRUBICIN FORMULATION

(71) Applicant: BELOTECA, INC., San Diego, CA (US)

(72) Inventors: Frederik Defesche, San Diego, CA (US); Dorla Mirejovsky, San Diego, CA (US); Jenny Tran, San Diego, CA (US)

(73) Assignee: BELOTECA, INC., San Diego, CA (US)

(21) Appl. No.: 19/402,231

(22) Filed: Nov. 26, 2025

Related U.S. Application Data
(60) Provisional application No. 63/747,650, filed on Jan. 21, 2025.

(51) Int. Cl. A61K 9/00; A61K 31/704; A61K 47/44

(52) U.S. Cl. CPC A61K 9/0034; A61K 31/704; A61K 47/44

(57) ABSTRACT

The invention is directed to a unit dosage vial containing 800 mg/20 mL of Valrubicin, a kit comprising the Valrubicin vial, and the use of such vial of Valrubicin in the treatment of cancer.

### CROSS-REFERENCE TO RELATED APPLICATIONS

This application claims the benefit of U.S. Provisional Application No. 63/747,650, filed Jan. 21, 2025, incorporated by reference herein in its entirety.

### FIELD OF THE INVENTION

This application relates to a unit dosage form to provide an 800 mg dose of Valrubicin at a concentration of 40 mg/mL. Valrubicin is useful in the treatment of bladder cancer.

### BACKGROUND OF THE INVENTION

Valrubicin is an anthracycline topoisomerase inhibitor indicated for intravesical therapy of BCG-refractory carcinoma in situ (CIS) of the urinary bladder in patients for whom immediate cystectomy would be associated with unacceptable morbidity or mortality.

The FDA approved commercial product VALSTAR® (valrubicin) solution for intravesical use is a sterile solution available in a carton of four 200 mg/5 mL single use 5-mL vials to provide the therapeutic dose of 800 mg/20 mL for this product. Thus, for each instillation, VALSTAR® is withdrawn from the four vials and diluted with 55 mL 0. 9% Sodium Chloride Injection, USP. The 75 mL of the then diluted VALSTAR® solution is instilled slowly via a urethral catheter into the bladder.

Therefore, a strength of valrubicin of 800 mg/20 mL would be fully consistent with the dosage and administration described in the labeling for the VALSTAR®. Moreover, the availability of an 800 mg/20 mL vial will provide convenience in the preparation of the recommended dose, decrease the chance for contamination, and reduce the amount of hazardous waste that is presently being generated using multiple vials. Thus, there is a need for an 800 mg/20 mL vial that is stable and has a degradation profile that is consistent with the FDA approved valrubicin product. The present invention satisfies that need.

### SUMMARY OF THE INVENTION

The present invention is based on the surprising discovery that, a headspace having a fill volume ratio of 0.5-0.6 will prevent excessive degradation of valrubicin in storage in a vial having 800 mg/20 mL.

Thus, in one embodiment, the present invention is directed to a valrubicin formulation comprising 800 mg valrubicin in 20 mL in a 50:50 solution of Polyoxyl castor oil:ethanol in a vial with fill volume ratio of 0.5-0.6 or an oxygen to fill volume ratio of 0.15-0.20.

In another embodiment, the present invention is directed to a valrubicin kit comprising the above formulation and instructions for administration.

In another embodiment, the present invention is directed to a method of treating a patient with bladder cancer, the method comprising administering the above formulation into the bladder of the patient.

### BRIEF DESCRIPTION OF THE DRAWINGS

FIG. 1 graphically depicts the Degradation Profile of VALSTAR® for 2 months at 25° C. Percent Impurity is plotted over time for each main degradant over the testing period.

FIG. 2 graphically depicts the Degradation Profile of Valrubicin Solution (800 mg/20 mL) in a 20 mL vial for 6 months at 25° C. Percent Impurity is plotted over time for each main degradant over the testing period.

FIG. 3 graphically depicts the stability of valrubicin filled in different vial sizes by showing related substances for vials stored for 6 months at 25° C. The 30 mL vial data is significantly different from the data of the other two sized vials.

### DETAILED DESCRIPTION OF THE INVENTION

The present invention is based upon the need for a single vial to provide 800 mg valrubicin at a concentration of 40 mg/mL. However, we have unexpectedly found that Valrubicin for Intravesical Instillation 40 mg/mL filled in 20 mL vials to yield the required therapeutic dose of 800 mg/20 mL was not stable. As the current 200 mg/5 mL strength is filled in 5 mL vials, it was assumed that one could proportionally scale up to 800 mg valrubicin in a 20 mL vial. Surprisingly, such scale up did not make a stable single vial product with a degradation profile that was consistent with the current 200 mg/5 mL strength. Instead, we have discovered that a 800 mg valrubicin in 20 mL, provided in a 30 mL vial, having a headspace of 0.5-0.6, provides a degradation profile similar to VALSTAR®. See Examples below.

### Definitions

The term “Polyoxyl castor oil” is polyethoxylated castor oil prepared by reacting ethylene oxide with castor oil in a ratio of 35:1. Commercially, it is commonly sold as Kolliphor or Cremophor.

The term “Valrubicin” refers to (N-trifluoroacetyladriamycin-14-valerate), a semisynthetic analog of the anthracycline doxorubicin, is a cytotoxic agent with the chemical name, (2S-cis)-2-[1,2,3,4,6,11-hexahydro-2,5,12-trihydroxy-7-methoxy-6,11-dioxo-4-[[2,3,6-trideoxy-3-[(trifluoroacetyl)amino]-α-L-lyxo-exopyranosyl]oxyl]-2-naphthacenyl]-2-oxoethylpentanoate.

The term “RLD” refers to the FDA approved VALSTAR® (Valrubicin solution for intravesical use) provided in four 200 mg/5 ml vials.

The term “fill volume ratio” means the volume of product divided by the volume of vial overflow capacity.

The phrase “oxygen to fill volume ratio” means ((1−Fill Volume Ratio)×(% Oxygen)/Fill Volume Ratio). The present application uses 20% oxygen in the calculations.

Thus, in some embodiments, provided is a valrubicin formulation comprising 800 mg valrubicin in 20 mL of a 50:50 solution of Polyoxyl castor oil:ethanol in a vial with fill volume ratio of 0.5-0.6 or an oxygen to fill volume ratio of 0.15-0.20. As discussed above and demonstrated in the Examples below, this formulation shows an amount of degradation impurities comparable to VALSTAR®. In some of these embodiments, the fill volume ratio is 0.5-0.6. In other embodiments, the formulation is in a 30 mL vial.

Also provided is a valrubicin kit comprising the above formulation and instructions for administration. In one aspect, the valrubicin is provided in a 30 mL vial. In another aspect, the kit further comprises a catheter for use in the administration.

Additionally provided is a method of treating a patient with bladder cancer. The method comprises administering the above formulation into the bladder of the patient. In some embodiments, 800 mg valrubicin is administered. In additional embodiments, the valrubicin formulation is in a 30 mL vial.

### General Experimental Methods

VALSTAR® (valrubicin) solution for intravesical use, 200 mg/5 mL, is available in 5 mL/13 mm Glass Type I Vials. It is supplied as a nonaqueous solution. Each vial of VALSTAR® contains 200 mg valrubicin at a concentration of 40 mg/mL in 5 mL of 50% Polyoxyl castor oil/50% dehydrated alcohol (ethanol), USP without preservatives or other additives. It is stored at 2° to 8° C. (36° to 46° F.).

The chemical structure of Valrubicin is shown below:

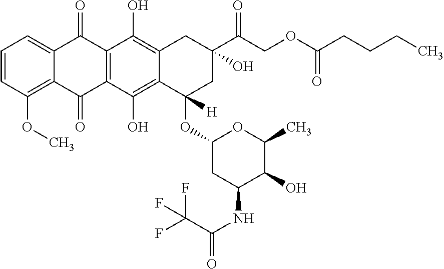 [SMILES: CCCCC(=O)OCC(=O)[C@]1(O)Cc2c(O)c3c(c(O)c2[C@@H](O[C@H]2C[C@H](NC(=O)C(F)(F)F)[C@H](O)[C@H](C)O2)C1)C(=O)c1c(OC)cccc1C3=O]

Analytical testing showed that VALSTAR®'s major degradants are doxorubicin valerate and one specified unidentified related compound with RRT 0.69-0.70.

The main known degradants of Valrubicin are shown in Table 1.

TABLE 1
Known Valrubicin Degradants

| | |
|---|---|
| Doxorubicin Valerate | 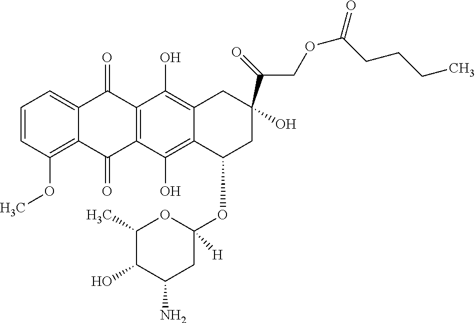 [SMILES: CCCCC(=O)OCC(=O)[C@]1(O)Cc2c(O)c3c(c(O)c2[C@@H](O[C@H]2C[C@H](N)[C@H](O)[C@H](C)O2)C1)C(=O)c1c(OC)cccc1C3=O] |
| Doxorubicinone Valerate | 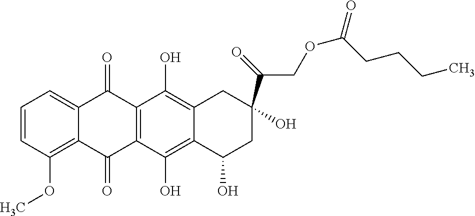 [SMILES: CCCCC(=O)OCC(=O)[C@]1(O)Cc2c(O)c3c(c(O)c2[C@@H](O)C1)C(=O)c1c(OC)cccc1C3=O] |
| Dianhydrovalrubicin | 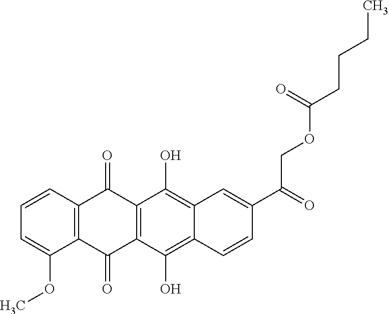 [SMILES: CCCCC(=O)OCC(=O)c1ccc2c(O)c3c(c(O)c2c1)C(=O)c1cccc(OC)c1C3=O] |

Valrubicin solution for intravesical use was tested using an internal high-performance liquid chromatography (HPPLC) method with configuration summarized in Table 2.

TABLE 2
HPLC Conditions for Valrubicin Testing
Column:
Phenomenex Kinetex 2.6 μm 100 Å Biphenyl 100 × 3.0 mm Column, PN 00D-4622-Y0
Solutions

| | |
|---|---|
| Mobile Phase A | 3.4 g/L Potassium Phosphate Monobasic, pH 3.1 |
| Mobile Phase B | 100% Methanol |
| Diluent | 50:50 ACN:Mobile Phase A Buffer |

Instrument Settings

| | |
|---|---|
| Flow Rate | 0.5 mL/min |
| Column Temperature | 40° C. |
| Injection Volume | 10 μL |
| UV Wavelength | 290 nm |
| Autosampler Temp. | 5° C. |

Gradient

| Time (minutes) | % Mobile Phase A | % Mobile Phase B |
|---|---|---|
| 0 | 80 | 20 |
| 8 | 40 | 60 |
| 20 | 20 | 80 |
| 32 | 12 | 88 |
| 33 | 10 | 90 |
| 37 | 10 | 90 |
| 38 | 80 | 20 |
| 42 | 80 | 20 |

### Preparation of Valrubicin Solution for Intravesical Instillation

The required amount of Polyoxyl 35 Castor Oil is mixed with the required amount of dehydrated alcohol (EtOH). While mixing add Valrubicin and continue mixing to obtain red solution without any visible particles.

### Example A: VALSTAR® Degradation Pathway

### 1. VALSTAR® Stability in 13 mm 5 mL Vial

The effect of VALSTAR® stability was investigated as part of the preformulation development of the 800 mg dose vial. The vials were stored at 25° C. for 2 months.

The stability data for VALSTAR® vials are shown in Table 3 and Table 4.

TABLE 3
Stability Results for VALSTAR ® Lot F118F009

| TEST | T = 0 | 1 MONTH | 2 MONTHS |
|---|---|---|---|
| Appearance | Clear, red solution with no | Clear, red solution with no | Clear, red |
| | visible particulates | visible particulates | solution with no |
| | | | visible |
| | | | particulates |
| Valrubicin Assay, | 100.9% | 105.9% | 99.5% |
| % Label Claim (40 mg/mL) | | | |
| 1) Specified-Identified | | | |
| Degradation Product | a. 0.23% | a. 0.25% | a. 0.22% |
| a. Doxorubicin Valerate | b. < LOQ | b. < LOQ | b. < LOQ |
| b. Doxorubicinone Valerate | c. < LOQ | c. < LOQ | c. < LOQ |
| c. Dianhydrovalrubicin | | | |
| 2) Specified-Unidentified | 0.14% | 0.29% | 0.34% |
| Degradation Product | | | |
| a. RRT 0.69-0.70 | | | |
| 3) Any Other Unspecified | 0.05% | 0.10% | 0.05% |
| Degradation Product | | | |
| 4) Total Related Substances | 0.42% | 0.64% | 0.61% |

TABLE 4
Stability Results for VALSTAR ® Lot F119E021

| TEST | T = 0 | 1 MONTH | 2 MONTHS |
|---|---|---|---|
| Appearance | Clear, red solution with no | Clear, red solution with no | Clear, red |
| | visible particulates | visible particulates | solution with no |
| | | | visible |
| | | | particulates |
| Valrubicin Assay, | 103.5% | 108.4% | 101.5% |
| % Label Claim (40 mg/mL) | | | |
| 1) Specified-Identified | | | |
| Degradation Product | a. 0.15% | a. 0.21% | a. 0.27% |
| a. Doxorubicin Valerate | b. < LOQ | b. < LOQ | b. < LOQ |
| b. Doxorubicinone Valerate | c. < LOQ | c. < LOQ | c. < LOQ |
| c. Dianhydrovalrubicin | | | |
| 2) Specified-Unidentified | < LOQ (0.04%) | 0.15% | 0.25% |
| Degradation Product | | | |
| a. RRT 0.69-0.70 | | | |
| 3) Any Other Unspecified | 0.18% | 0.19% | 0.18% |
| Degradation Product | | | |
| 4) Total Related Substances | 0.32% | 0.55% | 0.70% |

2. Valrubicin Solution for Intravesical Instillation 800 mg/Vial Filled in a 20 mL Vial

We prepared 800 mg/20-mL Valrubicin in a 20-mL vial. A new batch was prepared and filled as a 20 mL fill in a 20 mL vial to investigate whether a similar degradation result will be observed as for VALSTAR®. The vials were placed at 25° C. for up to 6 months in an inverted position.

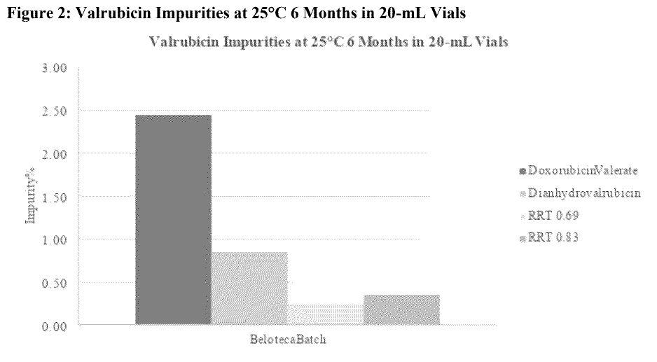

The test results obtained after 6 months are presented FIG. 2.

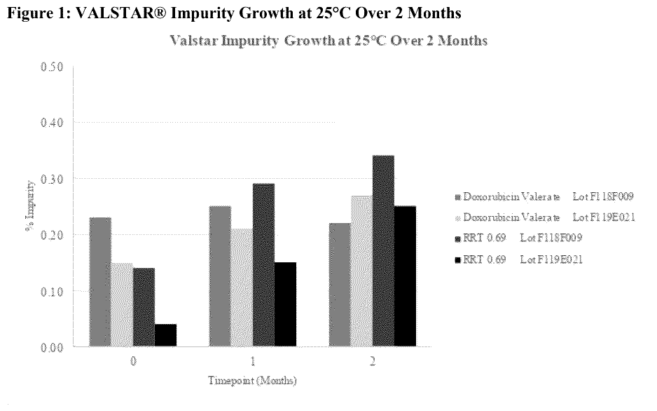

The pattern of degradation seen in the studies using VALSTAR® vials was not duplicated. More doxorubicin valerate and dianhydrovalrubicin were obtained than in VALSTAR® (FIG. 1). This unexpected result indicates that the ratio of the headspace to the fill volume in a 20 mL vial may not allow enough headspace to produce similar degradation as seen in VALSTAR®.

Therefore, we concluded that for the 800 mg/20 mL product a larger vial must be used to obtain the same degradation pathways as presented in Table 3 and Table 4 for VALSTAR®.

### Example B: Vial Size Study Optimization

The following experiment using different vial sizes was performed to show which sizes of vials would produce a stable formulation with a degradation pattern similar to the RLD VALSTAR® 200 mg/5 mL product. Various vials from 20 mL vial to 50 mL vials with approximately a 20 mL fill volume.

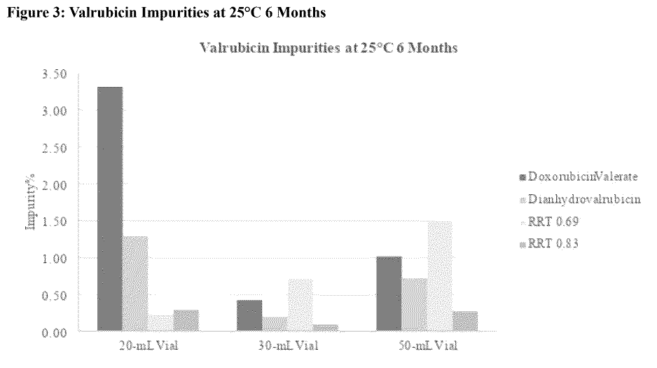

The vials were placed at 25° C. in inverted positions. The levels of main degradants of interest at 6 months are depicted in FIG. 3.

The testing of Valrubicin Solution for Intravesical Instillation filled in various vial sizes demonstrates that Valrubicin was the most stable when filled in a 30 mL vial. Valrubicin Solution for Intravesical Instillation filled in either a 20 mL or 50 mL vial showed more degradation than the solution filled in 30 mL vial. In addition, as shown in FIG. 3, the ratios of the main degradants were significantly different in the 20 mL and 50 ml vials compared to the 30 mL vial and VALSTAR®. The 30 mL vial showed the lowest amount of doxorubicin valerate and dianhydrovalrubicin, and exhibited the presence of specified unidentified related compound with RRT 0.69 with a very low amount of RRT 0.83. The amounts of these related compounds is close to the amounts of these degradation compounds found in VALSTAR®.

Table 5 shows fill volume ratios (defined as Target Fill Volume/Vial Overflow Capacity) and Oxygen to fill Volume Ratios (defined as (1−Fill Volume Ratio)×% Oxygen/Fill Volume Ratio) for various vial sizes.

TABLE 5
Evaluation of Various Vial Sizes

| Vial Format | 20 mL | 30 ML | 50 mL |
|---|---|---|---|
| Target Fill (mL) | 20.0-21.0 | 20.0-21.0 | 20.0-21.0 |
| Overflow | 26 | 38 | 62 |
| Capacity (mL) | | | |
| Target Fill/ | 0.77-0.81 | 0.53-0.55 | 0.32-0.34 |
| Vial Overflow* | | | |
| Oxygen to Fill | 0.06-0.05 | 0.18-0.16 | 0.43-0.39 |
| Volume Ratio** | | | |

*Fill Volume Ratio = Target Fill Volume/Vial Overflow Capacity
**Oxygen to Fill Volume Ratio = (1-Fill Volume Ratio) × % Oxygen/Fill Volume Ratio

In conclusion, the finding that degradation pathway of Valrubicin is dependent on the headspace was unexpected. Furthermore, it was unexpected that the size of the headspace changes how Valrubicin degrades and leads to very different degradation. In response to these findings, we established that 800 mg valrubicin at 40 mg/mL (20 mL) shows a degradation profile comparable to VALSTAR® when provided in a vial with a fill volume ratio of 0.5-0.6, for example a 30 mL vial.

All references cited herein, including patents, patent applications, and publications, are incorporated herein by reference, in their entirety. Whether previously specifically incorporated or not.

The various features and embodiments of the present invention, referred to in individual section above apply, as appropriate, to other sections, mutatis mutandis. Consequently, features specified in one section may be combined with features specified in other sections, as appropriate.

The foregoing description of some specific embodiments provides sufficient information that others can, by applying current knowledge, readily modify or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. In the drawings and the description, there have been disclosed exemplary embodiments and, although specific terms may have been employed, they are unless otherwise stated used in a generic and descriptive sense only and not for the purposes of limitation of the scope of the claims. Moreover, one skilled in the art will appreciate that certain steps of the methods discussed herein may be sequenced in alternative order or steps may be combined. Therefore, it is intended that the appended claims not be limited to the particular embodiment disclosed herein. Those skilled in the art will recognize, or be able to ascertain using no more than routine experimentation, many equivalents to the embodiments of the invention described herein. Such equivalents are encompassed by the following claims.

What is claimed is:

1. A valrubicin formulation comprising 800 mg valrubicin in 20 mL of a 50:50 solution of Polyoxyl castor oil:ethanol in a vial with fill volume ratio of 0.5-0.6 or an oxygen to fill volume ratio of 0.15-0.20.

2. The valrubicin formulation of claim 1, in a vial with a fill volume ratio of 0.5-0.6.

3. The valrubicin formulation of claim 1, in a 30 mL vial.

4. A valrubicin kit comprising the formulation of claim 1 and instructions for administration.

5. A valrubicin kit comprising the formulation of claim 3 and instructions for administration.

6. The kit of claim 4, further comprising a catheter.

7. The kit of claim 5, further comprising a catheter.

8. A method of treating a patient with bladder cancer, the method comprising administering the formulation of claim 1 into the bladder of the patient.

9. The method of claim 8, wherein 800 mg valrubicin is administered.

10. A method of treating a patient with bladder cancer, the method comprising administering the formulation of claim 3 into the bladder of the patient.

11. The method of claim 10, wherein 800 mg valrubicin is administered.
